(19) United States
(12) Patent Application Publication
(10) Pub. No.: US 2026/0199640 A1
(43) Pub. Date: Jul. 16, 2026

(54) A NON-OCCLUSIVE BALLOON CATHETER

(71) Applicant: Hoop Medical Limited, Ardcavan (IE)

(72) Inventor: Edward Charles Mudge, London (GB)

(21) Appl. No.: 19/139,316

(22) PCT Filed: Dec. 15, 2023
(86) PCT No.: PCT/IB2023/062772

§ 371 Date: Jun. 13, 2025

(30) Foreign Application Priority Data
Dec. 15, 2022 (ZA) 2022/13573

(51) Int. Cl. A61M 25/10

(52) U.S. Cl. CPC A61M 25/1002; A61M 2025/1004; A61M 2025/1072; A61M 2025/1084

(57) ABSTRACT

The invention provides a balloon catheter or medical device which includes a catheter tube defining a longitudinal axis, an inflation passage defined within the catheter tube and having an outlet, a flexible envelope which engages the catheter tube at least at a proximal end section and a distal end section to envelope the outlet to provide at least one balloon component which is inflatable, by inflow of an inflation fluid through the outlet, from a deflated state to an inflated state. In the inflated state, the balloon component is adapted or confined to provide one or more lobes and one or more channels, with a singular channel defined between adjacent sections of a singular lobe or each channel of a plurality defined between an adjacent pair of lobes. The channels provided the balloon with a unique shape, one of the applications of which is to provide a non-occlusion passage through which a bodily fluid can pass when the balloon catheter is deployed within a vessel.

### FIELD OF THE INVENTION

This invention relates to a balloon catheter and more particularly to a balloon catheter with a balloon module which includes lobes and channels, and which can be compliant, semi-compliant or non-compliant.

### BACKGROUND TO THE INVENTION

Medical balloon components are included in many diverse types of medical devices to treat a broad variety of medical conditions and these balloon components take in a range of shapes and forms.

Hereinafter, the term “component” and “module” are used interchangeably to refer to a discretely inflatable balloon unit. A balloon module means a balloon component which is made up of multiple sub-components into a balloon sub-assembly or “balloon module” which could then be assembled onto a medical device such as a balloon catheter or a robotically controlled surgical device.

A typical balloon catheter includes a balloon component which when inflated expands to do useful work within the human body via percutaneous access. The balloon catheter includes a balloon component which can be manipulated or actuated by means of fluid injection, between an un-inflated or contracted state in which state it has a relatively small diameter (such that it may be easily inserted into the human body through an introducer sheath within the groin for example) to and an inflated or deployed state, in which it has a larger diameter. The balloon component performs work in reaching a fully inflated or deployed state. For example, the balloon component can exert a radially outward force onto the inner portion of a blood vessel or a compressed/crimped metal stent or implant to dilate said implant into its deployed state. Examples of these more typical balloon catheters include PTA (Percutaneous Transluminal Angioplasty) balloon catheters, Foley balloon catheters and TAVI (Transcatheter Aortic Valve Implantation) balloon catheters.

Balloon components are sometimes subjected to a Pleat and Wrap process whereby longitudinal pleats or folds are induced to the balloon component and it is then wrapped around the catheter shaft, to minimise the crossing profile of the balloon in the un-inflated or contracted state.

Balloon components are also sometimes used on introducer sheaths/ports like for example the Kii™ balloon blunt-tip access system by Applied Medical which makes use of a balloon component to provide anchorage with the human body during laparoscopic procedures.

On many devices including a balloon component or balloon component assembly, the balloon component is mounted near to or at the distal end of the catheter shaft, However, the balloon component can also be mounted at any mid-way point along the shaft as is the case with the Kii™ Access System by Applied Medical. The catheter shaft usually runs some length (usually between 10 cm and 120 cm) and on or near the proximal end of the catheter shaft is an inflation port. This inflation port is in fluid communication with the balloon component. The catheter shaft often includes a guidewire lumen and/or a working channel as is appreciated by those familiar with devices within the minimally invasive medical devices industry.

The balloon component is inserted into the human body in an un-inflated state. With the smaller diameter, a smaller incisions or access route can be made with obvious medical advantages. The balloon component is connected to a means of inflation (such as a syringe or an auto-inflator) via an inflation lumen often running the entire length of the catheter. When the balloon component has been advanced into position, the means of inflation is actuated and the pressure within the inflation port, the inflation lumen, and the balloon component increase. This pressure increase is used to do work. The work, with respect to a “compliant balloon” component, may be to occlude a vessel or orifice (e.g., Foley catheters), and, with respect to a “non-compliant,” may be to deploy a stent or to dilate a stenosed artery. An example for this application is a PTA catheter.

Many balloon catheters remain in the body for only a brief time. PTA, PTCA (Percutaneous Transluminal Coronary Angioplasty) and TAVI balloon catheters are examples that typically remain in the body for a matter of minutes. Other balloon catheters, like Foley catheter or Intra-Aortic Balloon Pump (IABP) catheters, can remain in the human body for several weeks or even months.

Balloon catheter designers and engineers often refer to “compliant,” “semi compliant” and “non-compliant” balloon components. These three groups are intended to describe the way in which the balloon component changes in diameter as it is inflated to greater and greater pressures or as it is inflated with greater and greater volumes of inflation fluid (for example saline or air).

Compliant balloon components increase significantly in diameter as they are inflated. Compliant balloon components are generally made from relatively soft and elastic polymer or rubber materials with shore durometer hardness in the 20A to 90A range, such as silicones, latex, thermoplastic polyurethanes (TPUs) or thermoplastic elastomers (TPEs). These elastic materials can elongate or strain significantly as more inflation fluid is added within the balloon component. Balloon components made from these relatively soft materials typically inflate to take on a largely rounded, spherical or long-spherical shape when inflated to pressures greater than around 3 psi to 20 psi. Compliant balloons typically inflate at fairly low pressures in the 3 psi to 20 psi range. Indeed, some of the materials used for these types of balloon components have ultimate elongation values of around 500% (like TPUs) to around 1500% (like TPEs and Latex). Hence a compliant balloon can grow in diameter from say 3 mm in the un-inflated state to say 20 mm in the inflated state without rupture and at a relatively low inflation pressure. This feature can be useful for applications requiring occlusion or mechanical anchorage from the balloon component. It can also be useful for applications that require the balloon component to contact the target anatomy or device while exerting a relatively low force on that target anatomy or device to be deployed like for example a urinary balloon catheter like the Foley catheter.

Non-compliant balloon components on the other hand do not increase significantly in diameter as they are inflated to greater and greater pressures. The strong and relatively stiff materials from which these balloon components are made do not elongate or strain significantly as more inflation fluid is added within the balloon component. Consequently, the pressure within the balloon component increases significantly as more fluid is added, while the diameter of the balloon does not increase significantly. This can be useful for applications such as dilation of the target anatomy or device where a relatively high force (and high pressure) is required and a specific target diameter must be achieved, like for example in the deployment of a balloon expandable TAVI stented heart valve. Non-compliant balloon components are often made from relatively hard, in-elastic, and strong (high ultimate tensile strength) polymer materials with shore durometer hardness in the 70D to 90D range such as, for example, Polyethylene Terephthalate (PET) and Nylon 12 (PA12). Balloon components made from these relatively hard materials typically hold their shape and diameter when inflated to relatively high pressures in the 4 atm. to 40 atm. range.

Semi-compliant balloon components are those which fall into a group that is in between the compliant and non-compliant groups. Semi-compliant balloon components grow more in diameter than non-compliant balloon components when inflated to higher pressures. Semi-compliant balloons typically hold pressures that are in the range of 1 atm. to 20 atm. They can sometimes be configured to provide growth at relatively high pressures and can thus be used to provide a “one size fits all” type device, where the clinician can choose (usually within a fairly narrow range) the diameter to which they want to inflate the balloon, usually using a diameter vs pressure (or sometimes volume) chart or table provided by the device manufacturer within the instructions for use.

A standard shaped balloon component (like those used in PTA or PTCA procedures for example) can be described as having geometric features including a first or distal neck (a largely tubular or hollow cylindrical portion also known as a “leg” or “tail”) which transitions into a first or proximal conical portion (a conical or hemi-spherical region with a smaller diameter at the neck-to-cone transition and a larger diameter at the cone-to-mid-portion transition) which tapers wider to transition into a mid-portion or “working-length” of the balloon (which typically has an outer surface which is largely cylindrical in shape, and is region of the balloon component, when inflated, with the largest diameter). Distally, the mid-portion transitions to a second or distal conical portion which tapers narrower (or tapers down) to a second or distal neck which is largely tubular like the first neck.

The first and second necks are the portions of the balloon component that typically bond to the catheter shaft or shafts or to the medical device.

A conical balloon component has a shape like the standard balloon shape described above, but with no mid-portion or “working length.”

A spherical balloon component can be described as having two hemi spherical shaped cone sections connected to one another. So, for conical and spherical balloons, the conical portion can be described as the working length of the balloon since it is the only portion of the balloon that is not bonded or fixed to the catheter shaft.

A long spherical balloon component is like a spherical balloon component, but with a cylindrical working length or mid-portion positioned between the hemi spherical cones. In other words, a long spherical balloon component is like a standard shaped balloon component but with rounded cones or hemi-spherical cones.

For example, a compliant balloon component can change shape significantly between the un-inflated state and the inflated state. For example, a compliant balloon can have a standard shape when partially inflated (to some low pressure like 0.1 psi to 0.5spi) which then grows and changes into a spherical shape when fully inflated.

Dilation balloon components are often used to expand or open targets such as blocked or stenosed vessels or crimped-down metal stented implants for example. In some instances, the force needed to dilate the target is substantial and a high-pressure tolerant non-compliant balloon component is required.

Dilation balloons are often made from relatively hard, in-elastic, and strong (high ultimate tensile strength) polymer materials like those described as being “non-compliant” or “semi compliant” above. Balloons made from these relatively hard materials typically hold their shape when inflated to relatively high pressures in the 6 atm. to 30 atm. range, and because they can withstand these high pressures they are better suited to dilation applications which typically require moderate to high outward radial force from the balloon component.

Occlusion balloons are used to block or occlude vessels or orifices within the human body. Occlusion balloons often have an overall spherical or long spherical shape. The “cones” in these types of balloons are sometimes formed to have a conical shape when pressurised at very low pressures like 0.3 psi for instance. But due to the soft and compliant nature of the materials used for occlusion balloons, they typically lose their shape and “round out” when inflated to higher pressures (like 5 psi to 20 psi for example). A long-spherical balloon has a shape like a standard balloon but with rounded hemi-spherical “cones” on both ends of the working length. A spherical balloon typically has no cylindrical mid portion, but rather each cone transitions into the other directly.

Occlusion balloons are often made from relatively soft and elastic polymer or rubber materials as detailed in the compliant balloons' description above. Balloons made from these relatively soft materials typically grow to take on a largely round or spherical or long-spherical shape when inflated to pressures greater than around 5 psi to 20 psi. Said differently, an occlusion balloon does not typically hold its shape definition throughout inflation, but rather expands towards a rounder version of its original shape.

There are also examples of compliant balloons (typically made from relatively soft and elastic materials) which are used to dilate a target vessel, implant, or device, with relatively low outward radial force or pressure. This could be desirable to minimise the risk of damaging the target anatomy by exerting excessive dilation forces from the balloon. Since compliant balloons typically operate at low pressures, the forces exerted by these balloons are typically lower. The “Reliant” balloon by Medtronic and the “Coda” balloon by Cook are examples of compliant medical balloons used to remodel Aortic Stent Grafts. These balloons are designed to remove endo-leaks to treat Aortic Aneurysms with TEVAR (Thoracic Endo-Vascular Aortic Repair) or EVAR (Endo-Vascular Aortic Repair) procedures. An endo-leak is a failure of the stent graft to create a snug fit against the aorta at its proximal or distal end, thereby allowing a leak between the stent graft and the aortic-wall which can render the implant ineffective. Remodelling or dilating these stent grafts such that they are in contact with the aorta in a fully circumferential manner (allowing a pressure seal) requires a relatively low outward radial force and as such a compliant balloon can be used. Rather than low pressure dilation being unwanted (and as is the case with many TEVAR procedures) it can be desirable that the pressure within the balloon component remains low throughout its use to limit or remove the risk of damage to the surrounding anatomy.

There are also other more exotic balloon catheters fitted with balloon components with somewhat unusual shapes or configurations. For example, valvuloplasty balloon components (used to dilate or radially-expand heart valves) sometimes have an hourglass shape (where the working portion of the balloon includes a central “waist” region with a smaller diameter) that allows the balloon to fit more snugly within the target anatomy (a heart valve in this example)

Cryoablation balloon catheters are sometimes fitted with 2 balloon components, the inner balloon positioned radially inward from the outer balloon. In other words, the inner balloon is positioned within the outer balloon. The Arctic Front system is an example of a balloon catheter including two balloons as described here.

Some devices include balloon components which have an inverted or everted region. The Cook Cervical Ripening Balloon is an example of this. By inverting one end (for example the distal neck) of a balloon component, a favourable shape balloon assembly or balloon module can be achieved, and functionality of the medical device can be improved.

In practice the proximal neck of a standard shaped balloon component is sometimes larger in diameter than the distal neck to allow bonding of the proximal balloon neck to an outer catheter shaft and bonding of the distal balloon neck to a smaller diameter inner catheter shaft. The annular space between the inner and outer shafts is often used as the inflation lumen to inflate the balloon component. This type of catheter shaft configuration is sometimes referred to as a “coaxial” catheter shaft.

Those familiar with the art of balloon catheter design and manufacture will know that in other instances, the balloon component on a balloon catheter may have proximal and distal necks with the same or similar diameters and may be mounted on a multi-lumen catheter shaft which can accommodate the proximal and distal necks having similar diameters. One of the lumens in the multi-lumen shaft is used as an inflation lumen for the balloon component. The inflation lumen is exposed often by means of one or more “skives” or cuts or holes at a position that lies between the proximal and distal necks of the balloon component such that the inner portion of the balloon component is in fluid communication with the inflation lumen which is in fluid communication with the inflation port.

There are some less common balloon catheters which make use of a plurality of balloon components assembled in parallel such that the device allows perfusion through a vessel while the balloons are inflated there-in. Examples of these include the Bard True Flow™ balloon catheter which is used to pre-dilate the aortic valve prior to implantation of a TAVI valve; the Gore Tri-Lobe™ balloon catheter which is used to “remodel” or “post-dilate” a self-expanding stent graft within the thoracic abdominal aorta; and, more recently, the Disa Medinotec™ Trachealator Airway Dilation Balloon which is used to dilate the trachea. All of these devices are designed such that when inflated the balloon module or balloon assembly expands radially outward to dilate the target anatomy or target device by applying an outward radial force from within (much like a standard dilation type balloon) but whilst still allowing fluid or blood to flow through an at least partially hollow section of the inflated balloon module or balloon assembly, thus permitting perfusion during use.

A perfusion balloon device which includes multiple balloon components assembled in parallel (like the True-Flow & Tri-Lobe devices for example) has three key drawbacks, (i) the individual balloon components which make up the multi-balloon module are typically high value parts. On many balloon catheter devices, the balloon component may cost more than the other parts combined. So a design that relies on using multiple balloon components will typically have a significantly higher cost of parts which can be commercially undesirable. (ii) the additional assembly work needed to join each balloon component to the catheter shaft requires additional time and testing resulting in higher cost of development and manufacture (iii) The additional pressurised bonds may introduce additional risks which would need to be accounted for in the during the design verification phase of the product development, increasing the cost of the product's development.

By placing one standard shaped balloon component inside another and by including an inner support member positioned radially inward of both balloons a hollow balloon component that allows for perfusion while the balloons are inflated is realised. Such an invention is described in US20200179116.

### SUMMARY OF INVENTION

Hereinafter reference to “flexible envelope” means a flexible material i.e., the material from which the balloon module is composed.

Hereinafter, “base state” refers to the state of a balloon module, partially inflated to a pressure of between 0.1 and 1 psi, in which the balloon module takes on its inherent shape, with a minor diameter and a major diameter.

Hereinafter, the term “inflated state” denotes the condition of a balloon module being fully inflated to a specific pressure threshold, and within this threshold, the balloon maintains its inherent shape, but upon surpassing this pressure, it distorts from its inherent shape.

Hereinafter, the term “over-inflated state” denotes the condition of a balloon module overinflated past the pressure threshold, beyond which the balloon module distorts progressively from its inherent shape until the minor diameter equals the major diameter.

The invention provides a balloon catheter or medical device which includes:
a catheter tube defining a longitudinal axis,
an inflation passage defined within the catheter tube and having an outlet,
a flexible envelope which engages the catheter tube at least at a proximal end section and a distal end section to envelope the outlet to provide at least one balloon component which is inflatable, by inflow of an inflation fluid through the outlet, from a deflated state to an inflated state,
wherein, in the inflated state, the balloon component is adapted or confined to provide at least one lobe and at least one channel, wherein the at least one channel is formed between either adjacent sections of the at least one lobe or between an adjacent pair of lobes, and
wherein, at an outermost radial limit, the plurality of lobes defines a major (or working) diameter (OD), and, at an innermost radial limit, the plurality of channels define a minor diameter (ID).

The balloon catheter may be a non-occlusive balloon catheter.

The at least one channel and the at least one lobe may trace a spiral path that correspond to each other.

The balloon catheter may include a plurality of lobes and a plurality of channels.

Each lobe and each channel may extend in the longitudinal direction between at least the proximal end section and the distal end section.

The flexible envelope may engage the catheter along at least one intermediate section, between the proximal and the distal end sections, to configure the flexible envelope into at least a first balloon component and a second balloon component.

Each channel may extend directly between the proximal end section and the distal end section, alternatively, between the proximal end section and the intermediate section and the intermediate section and the distal section.

Alternatively, each channel may follow a tortuous, sinusoidal, or helical path between the respective sections.

The plurality of lobes may be uniformly angularly spaced relatively to one another, about the longitudinal axis.

Each channel and each lobe may be mutually co-extensive.

Alternatively, the channels may spiral in a clockwise and an anti-clockwise direction, intersecting at points to provide therebetween a plurality of diamond shaped lobes.

The flexible envelope may be made from a compliant or semi-compliant material (able to withstand mid-high pressures but which has more compliance and flexibility than a non-compliant material).

The material may comprise one or more of the following: a thermoplastic polyurethane (TPU), a thermoplastic elastomer, a silicone rubber, polyether block amide (PEBAX) and Nylon 12 (PA12).

Preferably, the flexible envelope is made from a homogenous material.

The balloon component may have, or be adapted with, a plurality of confined (or stiff or less compliant) sections, each confined section being at least partially co-extensive with a respective channel, and a plurality of expansive (or more compliant) sections, each expansive section at least partially co-extensive with a respective lobe.

In one alternative, the flexible envelope may be adapted in the confined sections with a thicker wall thickness when compared to the wall thickness in the compliant sections.

In the other alternative, adapted for higher pressure applications, such as for example vessel, tissue or implant dilation, the balloon catheter may include a tubiform brace which engages the outside of the flexible envelope, and which includes at least a proximal-end hub and a distal-end hub, and a plurality of longitudinally extending tensile elements, which extend between and connect the proximal-end and distal-end hubs.

The tubiform brace may include at least one intermediate hub, between the proximal-end and distal-end hubs and wherein the plurality of longitudinally extending tensile elements extend between and connect the proximal-end hub to the at least one intermediate hub and the at least one intermediate hub and the distal-end hub, respectively.

The plurality of elements may be uniformly angularly spaced.

Each element may extend along a respective confined section, adapted to confine that section in the inflated state to provide a respective channel as adjacent expansive sections inflate through a respective gap (or slot) between elements to provide respective lobes.

The brace may be made from a tubular blank which is cut to provide the hubs, elements, and the gaps.

The tubular blank may be made of a metal, a polymer, or a composite material.

Alternatively, the brace may be made from filaments which are formed or moulded to provide the hubs, elements, and gaps.

The filaments may be made from a suitable metal, polymer, or composite material.

Each filament may be a single-length wire, braid, or strand, from which the hubs and elements are integrally formed to minimise or remove junction points.

Each hub may be adapted to withstand radial expansion and constitutes a circumferential section of the brace.

The filaments constituting the hubs are formed with a plurality of diametrically or radially expansive formations, each of which is adapted to enable the brace to expand diametrically or radially enabling assembly of the brace over the balloon component.

The expansive formations may be waveforms, the height of which is aligned in the longitudinal axis, and the width of which is aligned with the catheter tube's circumference.

The filaments constituting the elements may be formed with at least one longitudinally extensive formation which is adapted to enable the brace to lengthen longitudinally in reaction to the inflation of the balloon component.

The at least one longitudinally extensive formation is a waveform, the height of which is with the catheter tube's circumference, and the width of which is aligned in the longitudinal axis.

Preferably, each element has two longitudinally extensive formations, one at each end and positioned within the balloon component's cone regions.

With the balloon module in a base state, the ID may be between 20% and 80% of the OD.

With the balloon module in the inflated state, the ID may be between 20% and 80% of the OD.

With the balloon module in the overinflated state, the ID may increase relative to the OD. In this state, each channel may shallow progressively until the ID is 100% of the OD.

The balloon catheter or medical device may include a tubular sleeve, positioned radially outward from the balloon component, which encloses at least part of the working-length of a respective balloon component, and which is adapted to limit the radial expansion of the plurality of lobes (and the OD) or to protect the flexible envelope from puncture by a stenosed, a calcified vessel or an implant, for example.

The tubular sleeve may be made of a suitable in-elastic material of high tensile strength, such as, for example, Kevlar.

### BRIEF DESCRIPTION OF THE DRAWINGS

The invention is further described by way of examples, with reference to the accompanying drawings in which:

FIGS. 1A to 1C schematically illustrate various embodiments of a balloon catheter device with one or more balloon modules;

FIGS. 2A to 2D illustrate a first embodiment of the balloon catheter device;

FIGS. 3A to 3H illustrate a first embodiment of the device, from a variety of views;

FIGS. 4A to 4F illustrate a first embodiment of the device;

FIGS. 5A to 5D illustrate a wire brace engaged with a balloon component, in an un-inflated state, and alone;

FIGS. 6A to 6C illustrate the wire brace engaged with a balloon component, of the device in accordance with a second embodiment, with the component in an inflated state;

FIGS. 7A to 7C illustrate a third embodiment of the device, with an outer sleeve enclosing at least part of the balloon component;

FIGS. 8A to 8B illustrate several embodiments of the device, with each embodiment having a differently shaped balloon component;

FIGS. 9A to 9F illustrate another embodiment of the invention which includes a wire brace with diamond-shaped apertures;

FIG. 10A to 10F illustrate a further embodiment of the invention in which the brace includes a pair of hub enclosing jackets; and

FIGS. 11A to 11F illustrate a final embodiment of the invention which has a singular spiral channel and commensurate lobe.

### DESCRIPTION OF PREFERRED EMBODIMENT

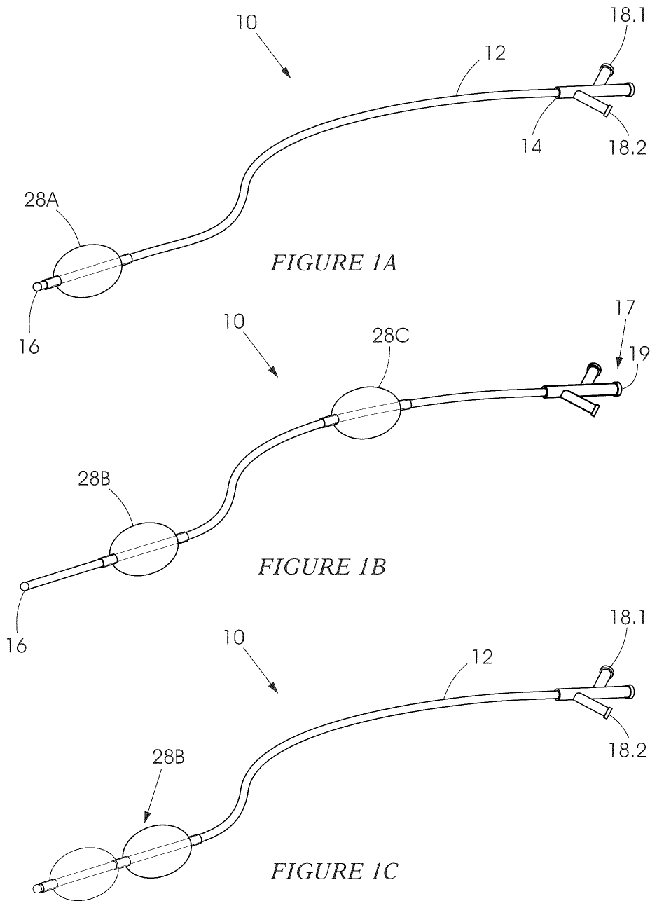

FIGS. 1 to 10 illustrate various embodiments and arrangements of a non-occlusive balloon catheter device 10 in accordance with the invention.

The balloon catheter device 10 includes an elongate, flexible catheter tube or shaft 12 which extends between a proximal end 14 and a distal end 16. On the proximal end, the catheter tube has a hub 15 which, in the illustrated example, has a pair of inflation ports, respectively designated 18.1 and 18.2, and working channel inlet port 19.

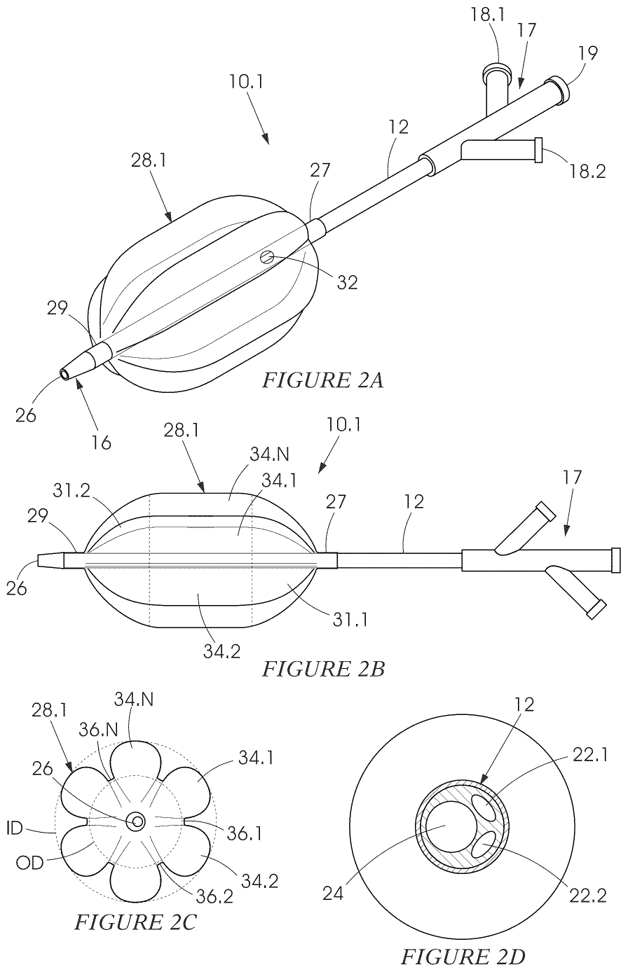

The inflation ports are in fluid communication with an inflation passage or passages 22.1 and 22.2 (see FIG. 2D). Also contained within tube 12 is a working passage 24 which terminates, at the distal end, at an opening 26.

Although a single inflation port may be sufficient, having two distinct inflation ports has the advantage in quicker inflation and deflation times and being able to cycle fluids through the ports, with one port as an inlet and one port as an outlet. The latter advantage would allow continuous substitution or cycling of the fluid within the balloon which could have application specific advantages like for example energy transfer during treatment.

As illustrated in FIG. 1A, on the distal end of the catheter tube 12, the device 10 has a single balloon component 28.A. In FIG. 1B, the device has two balloon components (respectively designated 28.B and 28.C) which are positioned mid-way between the proximal and distal ends (14, 16). In FIG. 1C, a double-balloon component embodiment 28.D, is illustrated, on the distal end. The balloon components can be configured in accordance with any of the embodiments described below.

It is however contemplated within the scope of the invention that a balloon component, or a plurality of balloon components, of various configurations, can be located on the catheter tube 12 at any point between the ends (14,16).

As for the structure and configuration of the balloon components, FIGS. 2A and 2B illustrate a first embodiment of the device 10.1 which balloon component 28.1 is composed of a flexible envelope, preferably made of a homogenous material, which is sealably affixed to the catheter tube 12 (drawn shorter for ease of illustration) at a circumferential proximal end section (proximal neck) 27 and a circumferential distal end section (distal neck) 29 to envelope an outlet (or outlets) 32 in the catheter tube.

For ease of explanation, only a single outlet 32, a single inflation passage and a single inflation port will be referred to, notwithstanding the presence of a plurality of these features in the embodiment being described.

The outlet 32 is in fluid communication, along the inflation passage 22, with the inflation port 18 to inflate the balloon component 28.1 by inflow of an inflation fluid through the outlet, from a deflated state to an inflated state. In this way, the deflated balloon component 28 makes it easy to insert them into a vessel and to manoeuvre the component into the target location where it can be inflated to perform useful work.

In the inflated state (as illustrated), the balloon component is adapted or confined (depending upon the embodiment, but in this embodiment the balloon component is adapted as will be described below) to provide a plurality of lobes (respectively designated 34.1, 34.2, . . . 34N) and a plurality of channels (respectively designated 36.1, 36.2, . . . 36N), with each channel defined between adjacent pairs of lobes (for example channel 36.1 which defined between lobe 34.1 and 34,2), and wherein, at an outermost radial limit, the plurality of lobes define a major (or working) diameter (OD) and, at an innermost radial limit, the plurality of channels define a minor diameter (ID). Both the OD and ID are illustrated on FIG. 2C with a dotted line.

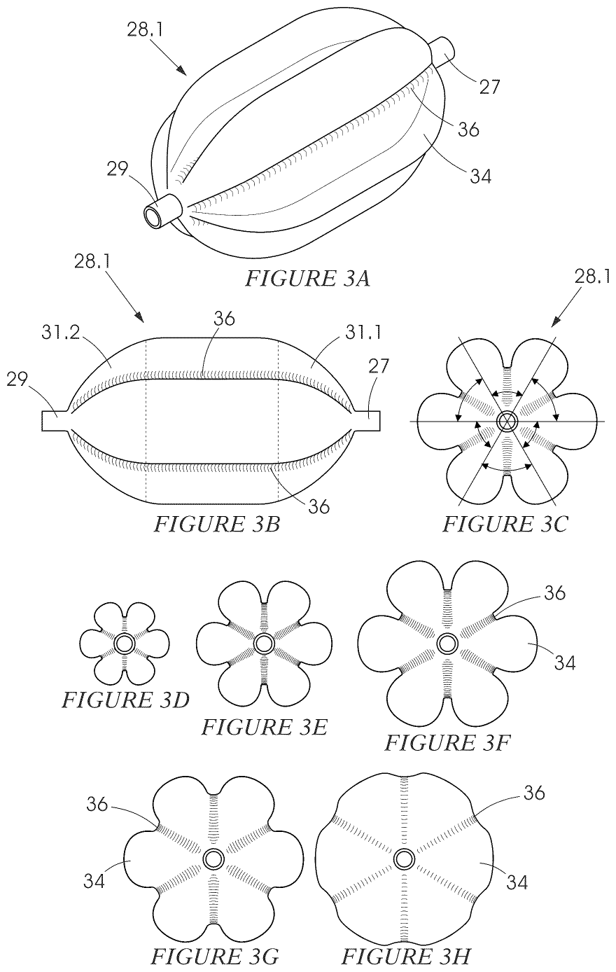

In this example, the channels, and the lobes, are longitudinally aligned and regularly angularly spaced relatively to the longitudinal axis. This is illustrated in FIG. 3C. The lobes are also substantially co-extensive with the channels, along the working length, with respective ends of each inflated lobe tapering, at respective cone sections 31.1 and 31.2, to the proximal and distal end sections (27, 29) respectively.

The material making up the flexible envelope of a balloon module may include one or more (in a blend or multi-layered) of the following: a TPU (a thermoplastic polyurethane) (usually 40A-75D shore durometer hardness); a TPE (a thermoplastic elastomer) (usually 15A-80A); PEBAX (by Arkema) (a polyether block amide) (usually 22D-75D); Nylon 12 (PA12) around 72D and PET (polyethylene terephthalate).

Examples of blends are TPU+PEBAX and PEBAX+PA12. A multi-layered flexible envelope can be made in a multi-layer extrusion process, allowing a balloon to be blown (requiring no additional blow forming steps) which also has an inner and an outer layer (bonded together everywhere as its co-extruded). Drug coating typically happens as a secondary process on the outer layer of the balloon.

The device 10 could also include radio-opaque marker bands (not drawn) positioned within the balloon component and or proximal and or distal of it to allow for positioning of the device within the target anatomy.

In FIGS. 3A, 3B and 3C, the balloon component 28.1 of the device 10.2 is shown in an inflated state, isometric, side view and end view. The series of FIGS. 3D to 3E sequentially illustrate the inflation cycle and how the OD and ID first both proportionally increase, until the OD reaches a limit (defined as a fully inflated state as illustrated in FIG. 3F), then the ID increases further, by a shallowing of the channels 36, as the inflation pressure is increased to an overinflated state (See FIGS. 3G and 3H).

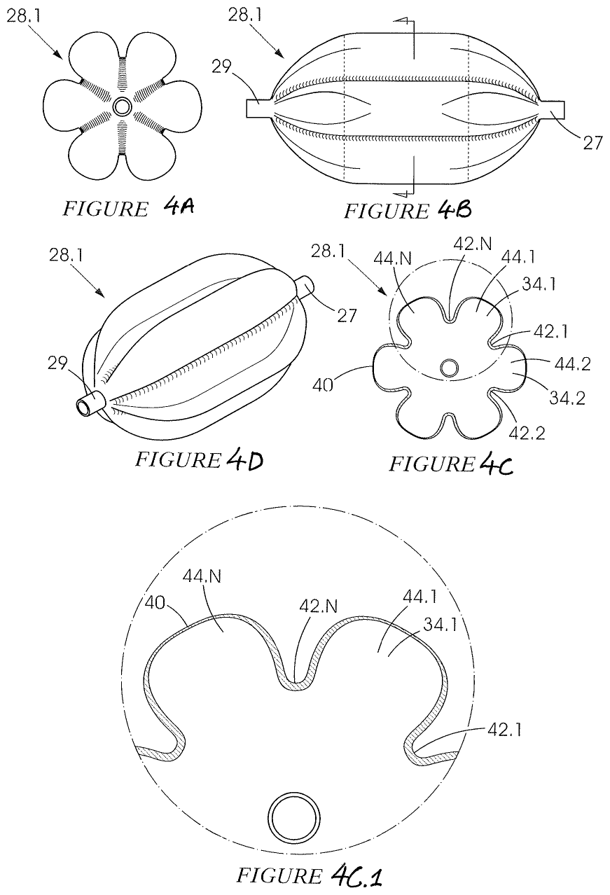

In FIGS. 4C and 4C.1, being cross-sectional views through a wall 40 of the balloon component 28.1, the flexible envelope is adapted with a plurality of stiff (or relatively less compliant) sections, respectively designated 42.1, 42.2, . . . 42.N, each confined section being at least partially co-extensive with a respective channel, and a plurality of expansive (or relatively more compliant) sections, respectively designated 44.1, 44.2, . . . 44.N, each expansive section at least partially co-extensive with a respective lobe.

In this example, the change in relative compliance between the stiff sections and the expansive sections is brought about by a change in the wall thickness of these sections. The stiff sections will have a thicker wall relative to the compliant sections. This is best illustrated in FIG. 4C.1.

To change the depth of the channels for a particular application, for any given inflation pressure, one or both of the following parameters of the flexible envelope may be changed: the material of manufacture, the wall thickness of the stiff sections and the wall thickness of the compliant sections.

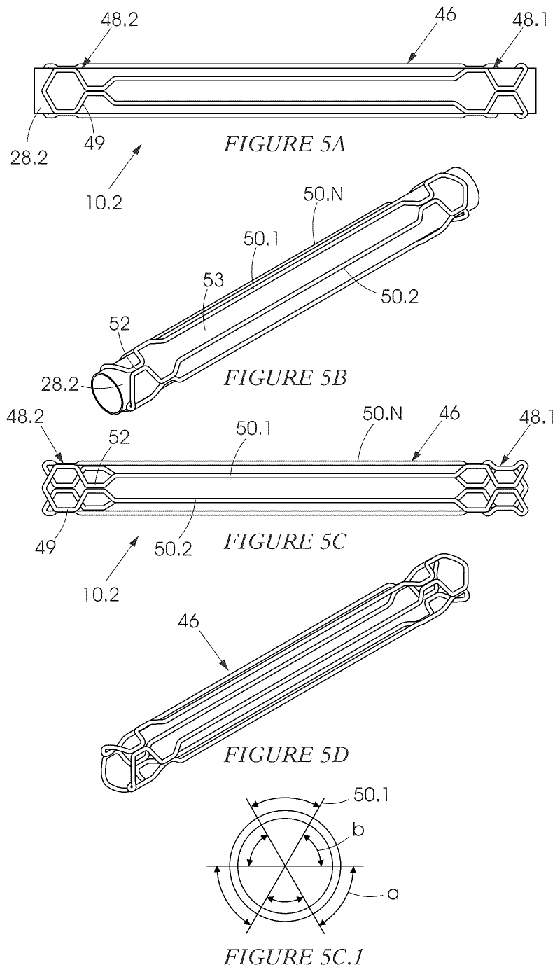

FIGS. 5A and 5B illustrate another embodiment of the invention in which the device 10.2 is adapted for higher pressure applications, such as for example vessel, tissue, or implant dilation. The device includes a tubiform brace 46 which is mounted over an un-inflated balloon component 28.2, positioned radially outwards from the component.

The brace 46 includes at least a proximal-end hub 48.1 and a distal-end hub 48.2, and a plurality of tensile elements (respectively designated 50.1, 50.2, . . . 50.N), which longitudinally extend between and connect the proximal-end and distal-end hubs. The plurality of elements is uniformly angularly spaced as is illustrated in FIG. 5C.1.

The brace 46 in this example is made with the hubs and the elements integrally formed to minimise or remove connection points which, in use, may rub against, and puncture, the flexible envelope. The brace can be made from a single length wire, braid, polymer strand, fibre-like carbon, Kevlar, or monofilament.

Within the hubs 48, the tensile elements 49 cross-link, along connection sections 52, to confer hoop strength and resist radial expansion, about the hubs, when the enclosed balloon component 28 inflates. However, they maintain the cylindrical shape of the hubs, but allow radial expansion in a controlled manner, one or more elements within the hub can be formed with a plurality of waveforms (not illustrated) with the amplitude (height) of the waveform aligned with the central axis of the catheter tube, and the wavelength (width) of the waveform aligned with the circumferential direction of the catheter tube, to enable the hub to diametrically expand. This can be functionally beneficial in enabling assembly over the compliant balloon component.

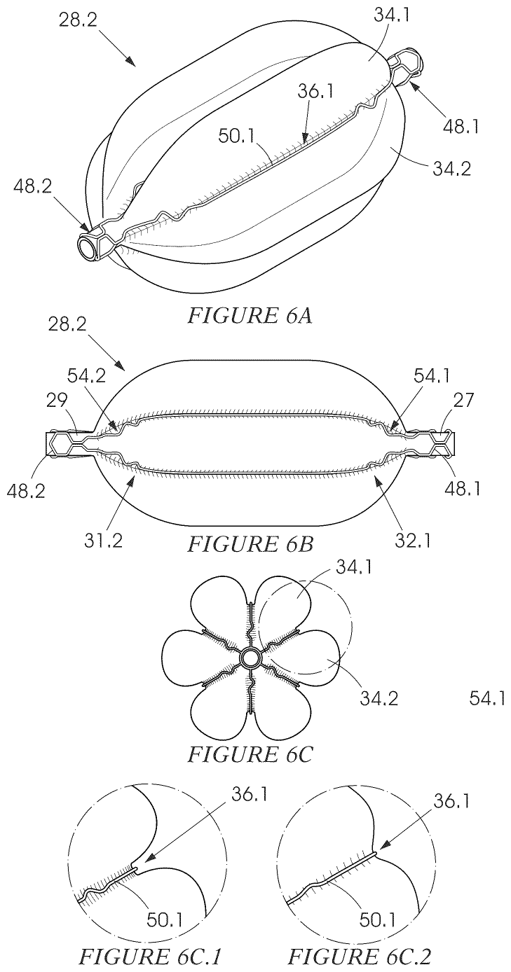

On engaging the balloon component 28, each element 50 extends along, and defines, a respective confined section 42. This is best illustrated in FIGS. 6A and 6C. On inflation of the balloon component, the element will confine this underlying confined section to provide a respective channel 36. And as adjacent expansive sections 44 inflate through a respective gap 53 between elements, respective lobes 34 are formed.

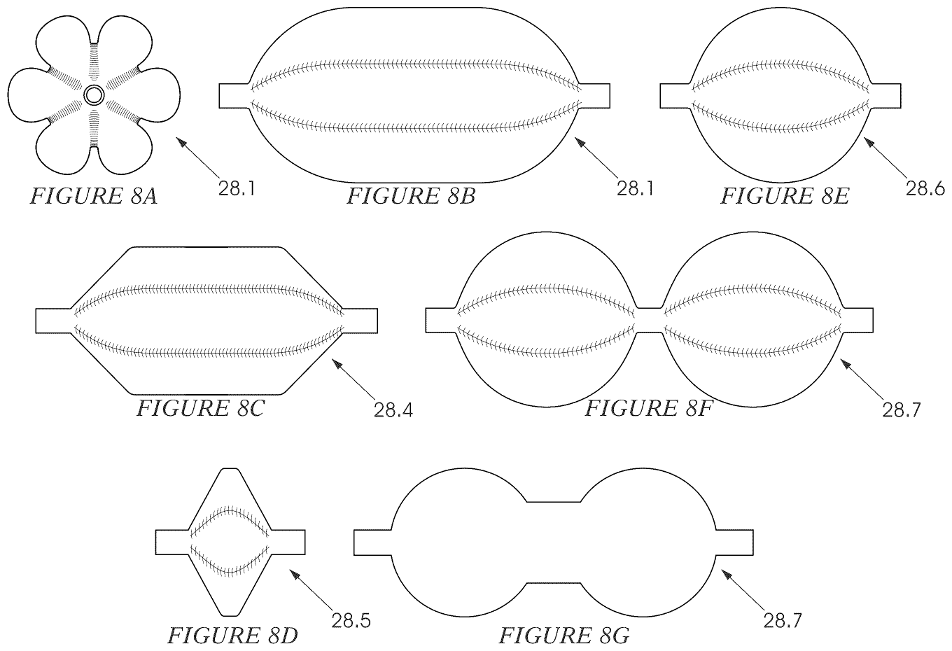

Although not illustrated, the tubiform brace can include at least one intermediate hub, between the proximal-end and distal-end hubs (48.1, 48.2) and wherein the plurality of longitudinally extending tensile elements extend between and connect the proximal-end hub to the at least one intermediate hub and the at least one intermediate hub and the distal-end hub, respectively. Such an adapted hub would confine an intermediate section of a balloon component 28 as illustrated in FIGS. 8F and 8G.

As illustrated in FIGS. 6A, 6B and 6C, each longitudinal tensile element 50 includes a longitudinally extensive section (54.1 and 54.2) at each end, positioned within the cone sections 31 of the balloon component. Each longitudinally extensive section is adapted to enable the frame or brace to lengthen longitudinally as a reaction to the inflation of the balloon component. This is best illustrated in FIGS. 6C1 to 6C3 which show an increasingly expanding balloon component and how the respective extensive section elongates to accommodate the expansion, allowing expansion of the ID, with an increase in inflation pressure. The longitudinally extensive section is formed into a plurality of waveforms (zig-zag sections) aligned with the amplitude of the waveform aligned with the circumferential direction of the catheter tube, and the wavelength of the waveform aligned with the long axis of the catheter tube.

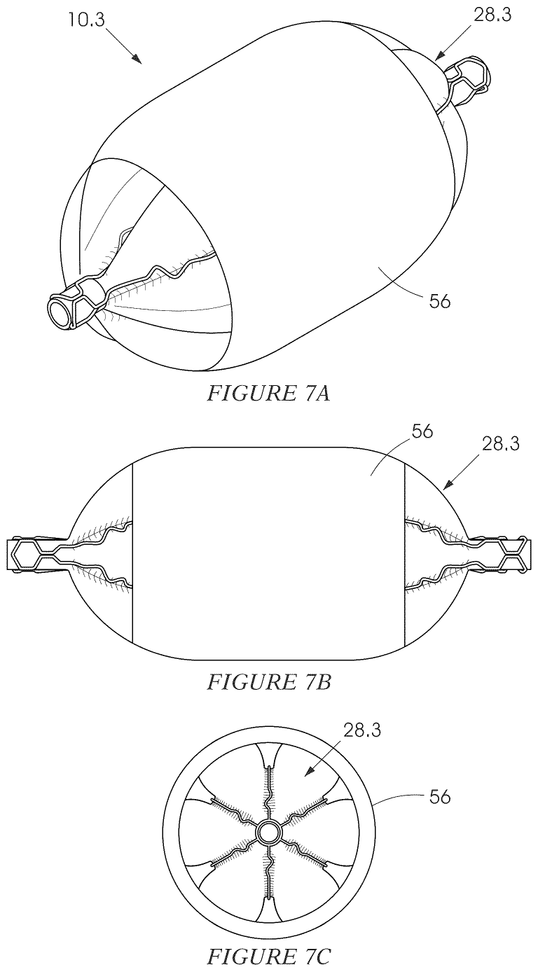

FIGS. 7A to 7C illustrate a balloon catheter device 10.3 in accordance with a third embodiment. In this embodiment, the device includes an outer tubular sleeve 56 positioned radially outward from the balloon component 28.3, along at least the component's working length. The balloon component of this embodiment is the same as the balloon component of the preceding embodiment.

The sleeve is configured to be in-elastic and to prevent the OD from increasing beyond the sleeve diameter. The sleeve can be made from a suitable material of high tensile strength such as Kevlar. The sleeve will protect the balloon component 28.3 from rupture or burst due when contacting sharp material like, for example, an implant or vascular calcium deposits.

FIGS. 8A to 8G illustrate a variety of preferred shapes of the balloon component (respectively designated 28.1 (the same shape as in the earlier described embodiments), 28.4, 28.5, 28.6 and 28.7). The shapes include long ovoid (28.1), long angular (28.4), diamond (28.5), spherical (28.6), and double spherical (28.7).

The double spherical embodiment can be formed with a suitably configured brace 46 such as that described above in paragraph 73. The number of balloon components however does not limit the invention and it is anticipated within the scope that there could be 1, 2, 3 . . . n, n+1 balloons.

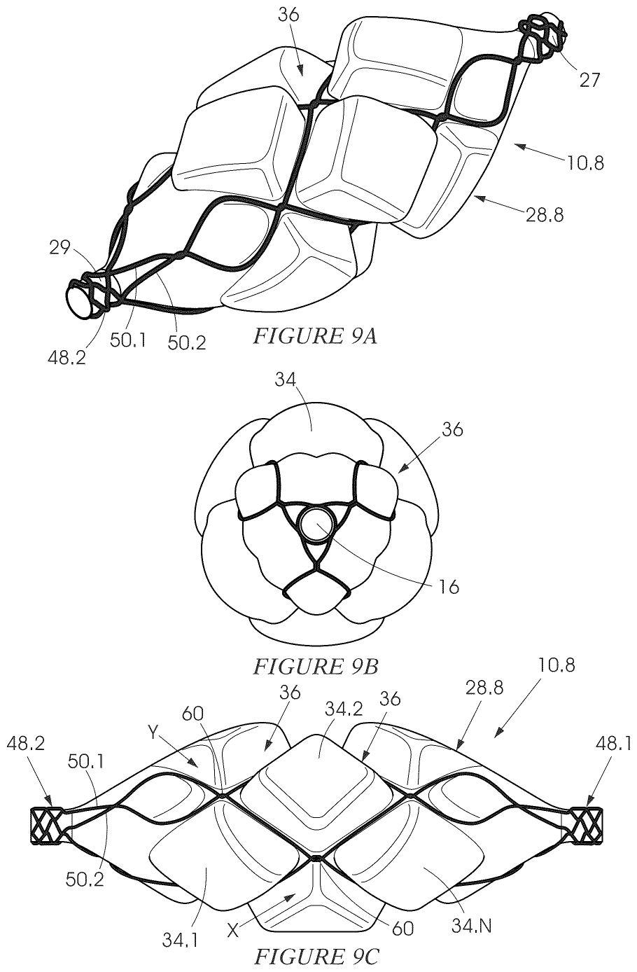

FIGS. 9A to 9C illustrate a preferred embodiment of the device 10.8 in which the tensile elements 50 of the brace 46 converge or criss-cross or join at one or more points 60 between the hubs, allowing perfusion between multiple diamond shaped lobes. This shapes the underlying balloon component 28.8 with a plurality of channels 36 which spiral in a clockwise (see directional arrow designated X) and an anti-clockwise (see directional arrow designated Y) direction, intersecting at the points. Diamond shaped lobes (34.1, 34.2 . . . 34N) protrude therebetween.

This diamond shaped embodiment 10.8 has numerous advantages. By connecting the tensile elements together between the hubs, the working length of the balloon component is better constrained within the brace, reducing the risk that the balloon component inflates into an undesirable shape by inflating more between one pair of tensile elements, inflating off to one side or “bull-frogging” as it is sometimes called. A brace with helical tensile elements which are not connected to other tensile elements, upon inflation of the balloon component, would induce undesirable torque on the catheter shaft.

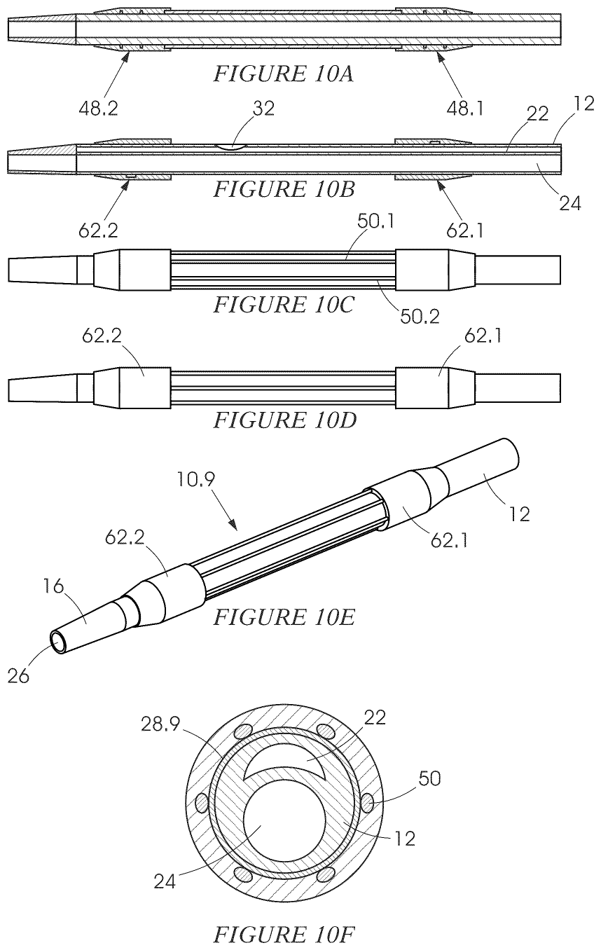

FIGS. 10A to 10F show another preferred embodiment of a balloon catheter device 10.9. In this embodiment, the brace 46.8 includes an outer jacket (respectively designated 62.1 and 62.2) positioned over each hub 48 of the brace 46.

This outer jacket 62 ensures that the respective hub 48 is frictionally engaged to the respective neck (27, 29) which, in turn, is held affixed to the catheter shaft 12, when the balloon component 28.9 is inflated.

This jacket can be made from a thermoplastic polymer like TPU or Nylon or Pebax and could be “re-flowed” or thermally bonded down onto the balloon component and or to the catheter shaft such that it envelops the hub of the brace.

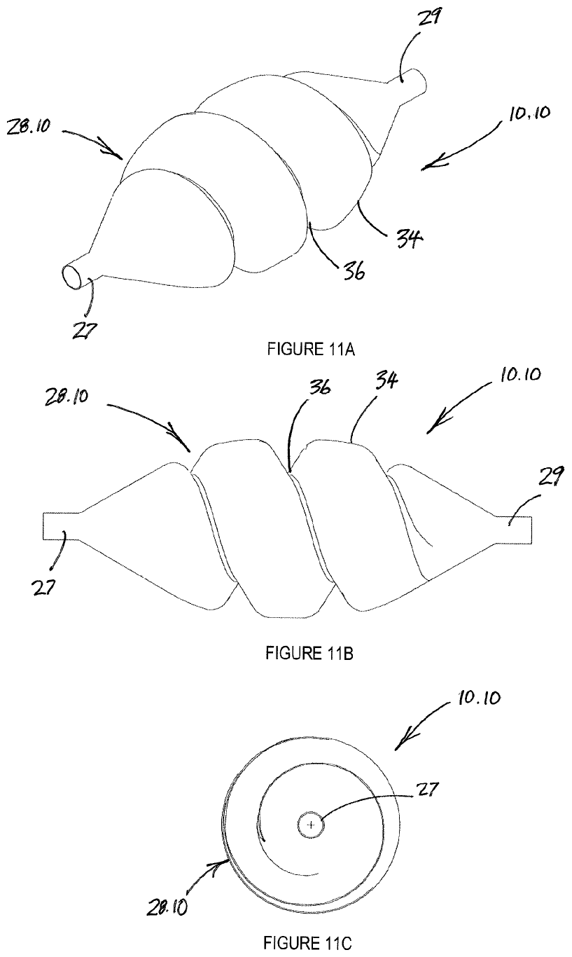

A final embodiment of the ballon catheter device 10.10 is illustrated in FIGS. 11A to 11C. What characterises this embodiment is that the balloon component 28.10 is formed with a single channel 36 which traces a spiral path from the proximal end section 27 to the distal end section 29. This single helical channel corresponds to a singular helical lobe 34.

The brace 46 of any of the embodiments can be made from a metal wire or from a high tensile strength polymer like “Ultra-high-molecular-weight polyethylene” or from a fibre like Kevlar or carbon fibre. Each tensile element can be mechanically connected to the others by means of winding one about the other, or by means of a bond, or a fastening or connecting component such as a ferrule or a sleeve. Alternatively, the tensile elements along with the hubs can be laser cut from a metal or polymer tube. At least a portion of the brace can be coated in a polymer. This could allow easier bonding of the brace to the balloon component. A brace coated in a soft polymer layer can also allow increased burst pressures as the soft polymer layer could cushion the brace on the balloon component, increasing the contact surface area between the brave and the balloon component and thereby reducing the stress in the balloon component and increasing the burst pressure.

The outer surface of the balloon component 28 can be coated in a drug, which would be beneficial as the drug could be delivered to the target anatomy in contact with the balloon, whilst still allowing perfusion of fluid (like blood or urine for example).

A compliant version of the invention includes the following functionality. The balloon component or flexible envelope includes a major diameter and a minor diameter. In a “base state” (defined as a low inflation pressure in the range of 0.1-1 psi) the minor diameter is significantly less than the major diameter. In an “inflated state” (defined as an inflation pressure greater than 2 psi and less than a “threshold pressure” defined below) both the major diameter and the minor diameter increase by a measurable amount compared to their respective measurement in the base state. In both the base state and in the inflated state the ratio between (or relative size of) the major diameter and the minor diameter can be described as follows:

$$1 / 5 Major Diameter \leq Minor Diameter \leq 4 / 5 Major Diameter$$

However, in a third state called the “over-inflated state” defined by an inflation pressure which exceeds a “threshold pressure” the:

$$Minor Diameter > 4 / 5 Major Diameter ;$$

Such that in the over-inflated state the minor diameter is approximately equal to the major diameter. This functionality is useful in applications where the user wishes to control the mass flow rate or volume flow rate of fluid passing through the perfusion channels. By increasing the inflation pressure, the user can reduce the flow rate in a controllable manner, effectively by reducing the cross-sectional area available for perfusion. By continuing to increase the inflation pressure even further, the user can fully occlude and arrest all flow as the minor diameter is made approximately equal to the major diameter. By reducing the inflation pressure, flow or perfusion can be restored again. This ability to control flow or even arrest flow, could be useful in applications such as REBOA (Resuscitative Endovascular Balloon Occlusion of the Aorta) for example.

1-35. (canceled)

36. A balloon catheter comprising:
a catheter tube defining a longitudinal axis, an inflation passage defined within the catheter tube and having an outlet;
a flexible envelope which engages the catheter tube at least at a proximal end section and a distal end section to envelope the outlet to provide at least one balloon component which is adapted to provide a plurality of confined sections and a plurality of expansive sections, and is inflatable, by inflow of an inflation fluid through the outlet, from a deflated state to an inflated state, wherein, in the inflated state, the balloon component provides a plurality of lobes with each lobe being at least partially co-extensive with a respective expansive section, along a working length, with respective ends of each lobe tapering at respective cone sections to the proximal and distal end sections respectively, and a plurality of channels, with each channel being at least partially co-extensive with a respective confined section and each channel is formed between a pair of lobes;
a tubiform brace which engages an exterior of the flexible envelope, and which includes at least a proximal-end hub and a distal-end hub, and a plurality of longitudinally extending tensile elements, which extend between and connect the proximal-end and distal-end hubs, and which extend along a respective confined section to confine the section in the inflated state to provide a respective channel and, as adjacent expansive sections inflate through a respective gap between elements, to provide respective lobes, wherein the brace is made from filaments which are formed to provide the hubs, elements, and gaps; and
wherein, at an outermost radial limit, the plurality of lobes defines a major diameter, and, at an innermost radial limit, the plurality of channels define a minor diameter.

37. A balloon catheter according to claim 36 wherein the lobes and the channels longitudinally extend between the proximal end section and the distal end section.

38. A balloon catheter according to claim 36 wherein the channels follows a tortuous, sinusoidal, or helical path between the proximal end section and the distal end section.

39. A balloon catheter according to claim 36 wherein the flexible envelope is made from a compliant material, a semi-compliant material, or a non-compliant material.

40. A balloon catheter according to claim 39 wherein the material comprises one or more of the following: a thermoplastic polyurethane, a thermoplastic elastomer, a silicone rubber, polyether block amide, polyethylene terephthalate and Nylon.

41. A balloon catheter according to claim 39 wherein the flexible envelope is made from a homogenous material.

42. A balloon catheter according to claim 36 wherein the plurality of lobes are uniformly angularly spaced relatively to one another, about the longitudinal axis.

43. A balloon catheter according to claim 42 wherein each channel and each lobe is mutually co-extensive.

44. A non-occlusive balloon catheter according to claim 36 wherein each hub constitutes a circumferential section of the brace, adapted to resist radial expansion.

45. A non-occlusive balloon catheter according to claim 36 wherein the plurality of elements are uniformly angularly spaced.

46. A non-occlusive balloon catheter according to claim 36 wherein the filament is a single-length wire, braid, or strand, with the hubs and the elements integrally formed to minimise or remove connection points.

47. A non-occlusive balloon catheter according to claim 36 wherein the filament is made from a metal, a polymer, or composite material.

48. A non-occlusive balloon catheter according to claim 36 wherein the filaments of the hubs are formed with a plurality of diametrically expansive formations which are adapted to enable the brace to diametrically expand enabling assembly over the compliant balloon component.

49. A non-occlusive balloon catheter according to claim 48 wherein the expansive formations are waveforms, the height of which is aligned in the longitudinal axis, and the width of which is aligned with the catheter tube's circumference.

50. A non-occlusive balloon catheter according to claim 36 wherein the filaments constituting the elements are formed with at least one longitudinally extensive formation which is adapted to enable the element to lengthen in reaction to the inflation of the balloon component.

51. A non-occlusive balloon catheter according to claim 50 wherein the at least one longitudinally extensive formation is a waveform, the height of which is with the catheter tube's circumference, and the width of which is aligned in the longitudinal axis.

52. A non-occlusive balloon catheter according to claim 50 wherein each element has two longitudinally extensive formations, one at each end, positioned within the balloon component's cone sections.
